(19) United States
(12) Patent Application Publication
(10) Pub. No.: US 2026/0248467 A1
(43) Pub. Date: Aug. 27, 2026

(54) SYSTEMS AND METHODS FOR COMPUTERIZED TOMOGRAPHY WITH LOW FREQUENCY ELECTROMAGNETIC RADIATION

(71) Applicant: ESI Imaging Inc., Kitchener (CA)

(72) Inventors: Omar Ramahi, Waterloo (CA); Mauricio Hernandez, Waterloo (CA); Hamid Akbari Chelaresi, Waterloo (CA)

(21) Appl. No.: 19/547,847

(22) Filed: Feb. 24, 2026

Related U.S. Application Data
(60) Provisional application No. 63/762,134, filed on Feb. 24, 2025.

(51) Int. Cl. A61B 6/03; A61B 6/00

(52) U.S. Cl. CPC A61B 6/032; A61B 6/4435

(57) ABSTRACT

Systems and methods for performing computerized tomography using low frequency electromagnetic radiation are disclosed. In one embodiment, a system for performing computerized tomography of an object under test using low frequency electromagnetic radiation comprises: a transmitter antenna configured to emit low frequency electromagnetic radiation towards the object under test; and a receiver comprising a metasurface having a plurality of receiver antennas configured to receive the low frequency electromagnetic radiation emitted from the transmitter antenna after passing through the object under test.

### CROSS-REFERENCE TO RELATED APPLICATIONS

This application claims priority to and the benefit of US Provisional Patent Application No. 63/762,134, filed on Feb. 24, 2025, the entire contents of which is incorporated herein by reference for all purposes.

### TECHNICAL FIELD

The present disclosure relates to computerized tomography, and in particular to computerized tomography using low frequency electromagnetic radiation.

### BACKGROUND

In principle, the penetration degree of waves and frequency of operation are inversely related to each other. Thus, to acquire sufficient penetration, lower frequency waves are desirable.

In medical imaging, for example, traditional computerized tomography (CT) scans use X-rays to perform soft tissue imaging. Traditional imaging methods using X-rays and CT scans have been the gold standards for many diagnostic applications. However, there are several problems and limitations associated with these imaging methods. Among other things, due to their reliance on ionizing radiation, there have been ongoing efforts to reduce exposure levels or develop alternative imaging techniques entirely. Further, X-ray-based imaging modalities also face challenges in producing high-contrast images, particularly when imaging dense biological tissues. Additionally, the cost of CT scans can be prohibitively expensive, both in terms of equipment costs and operational expenses.

Accordingly, additional, alternative, and/or improved systems and methods for computerized tomography remain highly desirable.

### SUMMARY

In accordance with one aspect of the present disclosure, a system for performing computerized tomography of an object under test using low frequency electromagnetic radiation is disclosed, comprising: a transmitter antenna configured to emit low frequency electromagnetic radiation towards the object under test; and a receiver comprising a metasurface having a plurality of receiver antennas configured to receive the low frequency electromagnetic radiation emitted from the transmitter antenna after passing through the object under test.

In some aspects, the metasurface is one dimensional or two dimensional.

In some aspects, the metasurface is two dimensional and and extends in a plane in parallel with a longitudinal axis of the object under test.

In some aspects, a height of the metasurface is greater than or equal to a height of the object under test in a longitudinal axis direction.

In some aspects, the transmitter antenna is linearly translatable with respect to the object under test in the longitudinal axis direction.

In some aspects, the low frequency electromagnetic radiation emitted by the transmitter antenna is recorded at a line of the plurality of receiver antennas that are in a plane comprising the transmitter antenna, the plane being perpendicular to the longitudinal axis of the object under test

In some aspects, the transmitter antenna and the receiver are rotatable around the object under test.

In some aspects, the object under test is rotatable with respect to the transmitter antenna and receiver.

In some aspects, the transmitter is configured to emit the low frequency electromagnetic radiation at a frequency of less than 1 GHz.

In some aspects, a size of each of the transmitter antenna and the plurality of receiver antennas is less than one-tenth a wavelength of the low frequency electromagnetic radiation.

In some aspects, a size of each of the transmitter antenna and the plurality of receiver antennas is less than one-tenth a radius of the object under test.

In accordance with another aspect of the present disclosure, a method for performing computerized tomography of an object under test using low frequency electromagnetic radiation is disclosed, comprising: positioning a transmitter antenna and a receiver on opposite sides of a longitudinal axis of the object under test in a first imaging position, wherein the transmitter antenna is configured to emit low frequency electromagnetic radiation towards the object under test; and the receiver comprises a metasurface having a plurality of receiver antennas configured to receive the low frequency electromagnetic radiation emitted from the transmitter antenna after passing through the object under test; and imaging the object under test at the first imaging position by: emitting the low frequency electromagnetic radiation from the transmitter antenna toward the object under test; and recording, at the plurality of antennas, the low frequency electromagnetic radiation received from the transmitter antenna after passing through the object under test.

In some aspects, the low frequency electromagnetic radiation is recorded at a line of the plurality of antennas that are in a plane comprising the transmitter antenna, the plane being perpendicular to the longitudinal axis of the object under test.

In some aspects, the method further comprises rotating the object under test about the longitudinal axis to a second imaging position, and performing the imaging of the object under test at the second imaging position.

In some aspects, the method further comprises rotating the transmitter antenna and the receiver about the longitudinal axis of the object under test to a second imaging position, and performing the imaging of the object under test at the second imaging position.

In some aspects, the method further comprises repeating the rotating and the imaging until the imaging is performed for 360 degrees about the longitudinal axis of the object under test.

In some aspects, the method further comprises translating the transmitter antenna in the longitudinal axis direction, and repeating the imaging of the object under test at further imaging positions for 360 degrees about the longitudinal axis of the object under test.

In some aspects, the method further comprises generating a profile of the object under test based on the low frequency electromagnetic radiation received at the plurality of antennas, at each imaging position.

In some aspects, the method further comprises generating a sinogram for each set of imaging positions in a plane perpendicular to the longitudinal axis direction.

In some aspects, the method further comprises reconstructing an image of the object under test based on the sinogram generated for each set of imaging positions.

### BRIEF DESCRIPTION OF THE DRAWINGS

Further features and advantages of the present disclosure will become apparent from the following detailed description, taken in combination with the appended drawings, in which:

FIG. 1 shows a representation of a four-layer cylindrical object as an object under test located between a transmitter and receiver;

FIGS. 2A and 2B show a schematic and a photograph of a fabricated strip-monopole antenna, respectively;

FIG. 3 shows a representation of a metasurface comprising an array of receiver antennas;

FIG. 4A shows a plot of a back-projected profile of the object under test shown in FIG. 1;

FIG. 4B shows a filtered sinogram of the object under test shown in FIG. 1;

FIG. 5 shows a reconstructed image of the object under test; and

FIGS. 6A and 6B shows a method for performing computerized tomography of an object under test using low frequency electromagnetic radiation.

It will be noted that throughout the appended drawings, like features are identified by like reference numerals.

### DETAILED DESCRIPTION

The present disclosure provides systems and methods for performing low frequency near-field imaging, and in particular to computerized tomography using low frequency (e.g. sub-1 GHz) electromagnetic radiation. In accordance with one aspect of the present disclosure, a system for performing computerized tomography of an object under test using low frequency electromagnetic radiation comprises a transmitter antenna that is configured to emit low frequency electromagnetic radiation towards the object under test, and a receiver comprising a metasurface having a plurality of receiver antennas configured to receive the low frequency electromagnetic radiation emitted from the transmitter antenna after passing through the object under test.

The design of both the transmitter and receiver antennas plays a crucial role in the overall performance of the system, particularly when operating at low frequency (e.g. sub-1 GHz frequencies), where designing compact and efficient antennas presents significant challenges. The transmitter antenna and receiver antennas should be both electrically and physically small. For example, a size of the transmitter and receiver antennas should generally be less than one-tenth a wavelength of the low frequency electromagnetic radiation (i.e. considered electrically small), and less than one-tenth a radius of the object under test (i.e. considered physically small). The systems and methods disclosed herein can be used to provide millimeter resolution below half wavelength limits, surpassing diffraction constraints, and allowing for sub-wavelength field sampling.

Further, to help address challenges with using electrically and physically small antennas, as described herein the receiver in accordance with the present disclosure comprises a metasurface with an array of receiver antennas, strategically incorporating a sufficient number of unit cells to enhance imaging resolution. Thus, unlike conventional scanning methods that sequentially examine different regions of the object under test, the approach in accordance with the present disclosure enables the simultaneous imaging of multiple unit cells within the metasurface. This parallel imaging capability drastically reduces the scanning time by reducing the number of rotations of the transmitter and receiver around the object under test required to fully image the object under test, improving efficiency while maintaining high-resolution imaging performance. Moreover, the metasurface may be two dimensional and extending in a plane in parallel with a longitudinal axis of the object under test, in some cases with a height that is greater than or equal to a height of the object under test, and therefore only the transmitter needs to be translated along the object under test to image the entire length of the object under test, further improving efficiency in the scanning process.

Advantageously, the systems and methods in accordance with the present disclosure utilize microwave imaging principles to create high-resolution images of biological tissues. Unlike traditional imaging methods such as X-ray and MRI, microwave or low frequency electromagnetic radiation imaging offers a non-ionizing, cost-effective, and potentially portable alternative. The use of low frequency electromagnetic radiation enhances penetration depth while maintaining sufficient resolution, making it particularly suitable for applications such as tomography and mammography.

The technology adapts the principles of computerized tomography (CT) to work with microwave radiation or low frequency electromagnetic radiation instead of X-rays. The systems and methods may use the Radon transform technique and other reconstruction algorithms to generate tomographic images from multiple projections. This adaptation allows for the development of a low frequency electromagnetic radiation based CT-like imaging system, offering a safe and effective alternative to traditional CT scans for soft tissue imaging.

The systems and methods in accordance with the present disclosure address several problems associated with traditional imaging modalities, particularly in the context of medical imaging, including but not limited to:

Limitations of Ionizing Radiation: Traditional imaging techniques like X-rays and CT scans often involve ionizing radiation, which poses health risks to patients. The systems and methods for performing computerized tomography using low frequency electromagnetic radiation disclosed herein eliminates this risk by using non-ionizing microwave or lower frequency electromagnetic radiation, providing a safer alternative for imaging.

Cost of Imaging Technology: Existing imaging modalities such as MRI and CT scans can be prohibitively expensive, both in terms of equipment costs and operational expenses. The use of low frequency electromagnetic radiation and inexpensive components in accordance with the present disclosure aims to make imaging technology more accessible and affordable.

Resolution and Image Quality: One of the challenges in microwave imaging has been the ability to generate high-resolution images comparable to those obtained from other modalities. The systems and methods for performing computerized tomography using low frequency electromagnetic radiation leverages the unique behavior of an ensemble of electrically small antennas, functioning as a metasurface sensor, to achieve high-resolution imaging, thereby improving the quality of diagnostic images.

Reconstruction Challenges in Microwave Imaging: Traditional microwave imaging techniques often lack reliable and simulation-free reconstruction approaches, leading to less effective imaging outcomes. In accordance with the systems and methods for performing computerized tomography using low frequency electromagnetic radiation, the Radon transform is used as the reconstruction methodology, which enhances reliability and can produce clearer images of the object under test.

Complexity of Interpretation: The varying dielectric and electromagnetic properties of different tissues can complicate the interpretation of microwave imaging results. By focusing on electrically small sources and their interactions, the systems and methods for performing computerized tomography using low frequency electromagnetic radiation simplifies this interpretation process, making it easier to understand the internal structures of the object under observation.

It will thus be appreciated that the systems and methods for performing computerized tomography using low frequency electromagnetic radiation in accordance with the present disclosure offer several notable advantages over traditional imaging methods and existing microwave imaging techniques, including but not limited to:

Non-Ionizing Radiation: The use of low frequency electromagnetic radiation eliminates the risks associated with ionizing radiation found in conventional X-ray and CT imaging. This makes it a safer alternative for patients, especially in scenarios requiring frequent imaging, such as cancer surveillance.

Cost Effectiveness: The systems and methods for performing computerized tomography using low frequency electromagnetic radiation leverages inexpensive components for radiation generation and detection, which significantly reduces the overall cost of the imaging system compared to high-end imaging technologies like MRI and traditional CT scans. This potentially makes advanced diagnostic imaging more accessible to a broader audience.

High Resolution: The system aims to achieve high-resolution images, potentially exceeding the capabilities of traditional microwave imaging modalities. The systems and methods for performing computerized tomography using low frequency electromagnetic radiation utilizes the unique properties of an ensemble of electrically small antennas, functioning as a metasurface sensor, to enhance resolution and image quality.

Scanning Time Improvement: Using an ensemble of electrically small antennas, functioning as a metasurface sensor reduces the scanning time compared to conventional imaging methods that rely on sequential scanning of individual points or regions. By enabling simultaneous imaging of all unit cells within the metasurface, this approach eliminates the need for step-by-step scanning, significantly improving efficiency. As a result, high-resolution images can be obtained in a much shorter time, enhancing the practicality of microwave imaging for real-time diagnostics and medical applications.

Simplified Image Reconstruction: By employing a reconstruction approach based on the Radon transform, the systems and methods for performing computerized tomography using low frequency electromagnetic radiation avoids complex, resource-intensive inverse scattering techniques commonly used in microwave imaging. This leads to more straightforward and reliable image reconstruction processes, improving the practical applicability of the technology.

Enhanced Object Interrogation: The systems and methods for performing computerized tomography using low frequency electromagnetic radiation focuses on the near-field interactions and utilizes electrically small antennas to create significant energy coupling between the source and the object under test (OUT). This allows for improved signal reception and clearer image formation by exploiting the ray-like behavior of low frequency electromagnetic radiation under specific conditions.

Ability to Penetrate Various Tissues: Low frequency electromagnetic radiation has better penetration capabilities compared to higher-frequency electromagnetic waves. This allows for effective imaging of different types of biological tissues without causing damage, which is especially beneficial in medical contexts.

Computational Efficiency: The imaging approach presented is computationally inexpensive, focusing on magnitude-only information from transmitted waves to reconstruct images. This aspect enhances the practicality of the technology, making it easier to implement in real-time applications.

Moreover, it will be appreciated that while the present disclosure contemplates use of computerized tomography with low frequency electromagnetic radiation in the context of medical imaging, there are a range of applications that the technology may be applicable to beyond medical imaging, including geographical remote sensing and through-wall imaging. Thus, its versatility makes it suitable for a variety of diagnostic and monitoring scenarios.

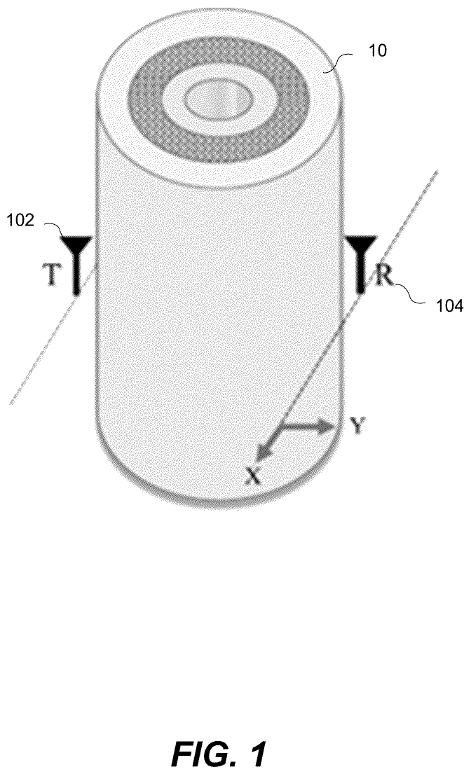

Embodiments are described below, by way of example only, with reference to FIGS. 1-6B.

FIG. 1 shows a representation of a four-layer cylindrical object as an object under test (OUT) 10 located between a transmitter antenna 102 and a receiver 104. The system is arranged with the OUT 10 placed in the scanning setup where the transmitter antenna 102 and the receiver 104 remain set to a predefined distance (e.g. 1 mm) away from the cylinder. The transmitter antenna 102 and the receiver 104 should be positioned close to the OUT 10 to allow for capturing signals within the near-field of the antennas, allowing for significant interaction between the low frequency electromagnetic radiation and the OUT, which is important for high-resolution imaging.

As described further herein, the receiver 104 comprises a metasurface having a plurality of receiver antennas. In an imaging configuration, the transmitter 102 and the receiver 104 are arranged on opposite sides of the OUT 10, i.e. opposite sides with respect to a longitudinal axis (perpendicular to the x-y plane) of the OUT 10. The transmitter 102 is configured to emit low frequency electromagnetic radiation (e.g. less than 1 GHz) towards the OUT 10, and the receiver antennas of the receiver 104 are configured to receive the low frequency electromagnetic radiation emitted from the transmitter antenna after passing through the OUT 10.

As described in “Toward Computerized Tomography with Microwaves” by Mirjahanmardi et al., IEEE Transactions on Microwave Theory and Techniques, Volume 70, NO. 11, November 2022, the entire contents of which is incorporated herein by reference in its entirety, if the OUT 10 is electrically small (appreciably smaller than the wavelength of the radiating source), then the impression/projection due to a localized source impacting the OUT will be highly similar to an impression/projection generated by a ray-like source. That is, the receiver 104 placed closest to the source will be impacted mostly by the composition of the object along the direct path between the transmitter 102 and the receiver 104. Therefore, the low frequency electromagnetic radiation energy interaction with the interrogated medium is highly similar to the way X-rays that interact with the medium in the sense that X-rays leave an impression on the film (i.e., the receiver or receiving point) that is directly dependent on the tissues in the straight path that the X-rays traverse between the source and a point on the film. This highly non-intuitive field behavior is important to computerized tomography using low frequency electromagnetic radiation because the reconstruction approach based on the Radon transform can be analogous to the one used in computerized tomography (CT) based on X-ray illumination

There are two important considerations: (1) the close proximity of the OUT 10 to the radiation source, and (2) using sufficiently low frequency to ensure full penetration of the OUT 10. Therefore, the interrogation of the OUT 10 must take place within the near field of the radiating source. The use of an electrically small microwave radiation source, which provides a ray-like radiation within the OUT, allows use of Radon transforms at low frequencies.

In accordance with the present disclosure, the transmitter antenna 102 and receiver antennas are configured using electrically small radiators (e.g. less than one-tenth a wavelength of low frequency electromagnetic radiation) while using a sufficiently low frequency (e.g. sub-1 GHz) that renders the entire OUT 10 electrically very small. The use of electrically small antennas is important because their size relative to the wavelength affects the propagation behavior of the low frequency electromagnetic radiation. These antennas enable effective transmission and reception of the low frequency electromagnetic radiation necessary for imaging and ensure high-resolution data collection.

The combination of small source size and low frequency creates a phenomenon whereby the effect on a receiving antenna largely depends on its proximity to the source. Thus, for the receiver 104 comprising a plurality of receiver antennas that is placed on the opposite side of the transmitter 102 (opposite with respect to the OUT 10), the energy collected by the receiver will be directly relevant to the OUT's “slice” that connects the transmitter to the receiver.

As described further herein, the OUT 10 may be scanned by systematically rotating both the transmitter antenna 102 and the receiver 104 at multiple angles by rotationally sweeping the antennas to capture data from various perspectives. Alternatively, it is possible that the OUT 10 may be rotated while the transmitter antenna 102 and receiver 104 are not rotated. At each imaging position, the transmitter antenna 102 and receiver 104 may be translated perpendicular to the longitudinal axis of the OUT 10 (e.g. in the x-axis direction) during the imaging process.

By configuring the receiver 104 as a metasurface comprising a plurality of receiver antennas, data indicative of the low frequency electromagnetic radiation received from the transmitter after passing through the object under test can be recorded for the plurality of receiver antennas at a time. In particular, as described further below, data may be recorded for a plurality of receiver antennas that are in a line in the same x-y plane as the transmitter antenna 102. Advantageously, this means that more data is captured at each imaging position, allowing for larger rotations of the transmitter/receiver between imaging, and thus improving efficiency and reducing imaging time.

After capturing data 360 degrees around the OUT 10 in a given x-y plane perpendicular to a longitudinal axis of the OUT, the transmitter antenna 102 and the receiver 104 may be linearly translated (i.e. in the longitudinal axis direction, perpendicular to the x-y plane), and the process repeated to fully capture data of the OUT 10 along the entire length of the OUT 10. In some embodiments, the receiver 104 may be a 2D array of antennas extending in a plane in parallel to the longitudinal axis of the OUT so it may not be necessary to linearly translate the receiver 104 each time. In still further embodiment, the receiver 104 may have a height that is greater than or equal to height of the OUT 10, so it is not necessary to linearly translate the receiver 104 at all.

A comprehensive data acquisition system (not shown) may be used to collect real-time signal data during the imaging process. The data acquisition system should accurately capture the signals from both the transmitter and receiver to allow for precise reconstruction of images, as described further herein.

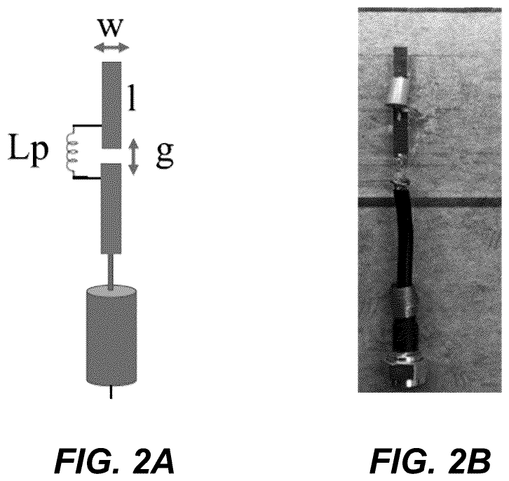

FIGS. 2A and 2B show a schematic and a fabricated of a fabricated strip-monopole antenna, respectively, which may be used as the transmitter antenna 102 shown in FIG. 1. As described above, the antenna is configured to emit a low frequency electromagnetic radiation source. A low frequency source is important for penetrating the OUT without causing ionization. The low frequency supports deeper penetration into biological or complex materials, making it suitable for applications like medical imaging.

As described above, it is important for the transmitter antenna to be both electrically and physically small. With reference to FIG. 2A, the parameters l, w, and g are 2 cm, 3 mm, and 1 mm, respectively. Accordingly, the size of the antenna (e.g. the width being 3 mm) is much less than one-tenth the wavelength of the low frequency electromagnetic radiation and much less than one-tenth the radius of the OUT.

While FIGS. 2A and 2B show a strip-monopole antenna, it will be appreciated that other types of antenna types could be utilized for the transmitter antenna, as well as for the receiver antennas. For example, other antenna types may include an electrically small loop antenna or arrays.

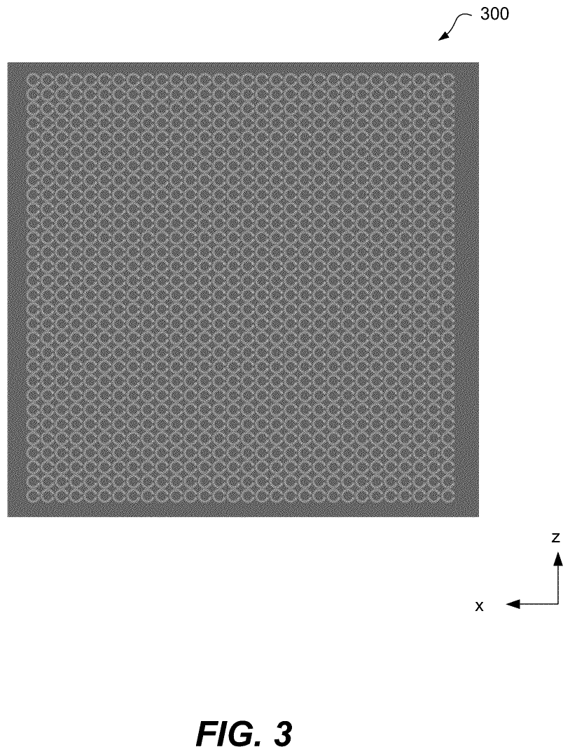

FIG. 3 shows a representation of a metasurface 300 comprising an array of receiver antennas, which may be used as the receiver 104 shown in FIG. 1. As described above, the receiver comprises a metasurface having an array of electrically small antennas.

As shown in FIG. 3, the metasurface 300 is 2-dimensional, and extends in an x-z plane in parallel with the longitudinal axis of the OUT. The metasurface 300 provides a film-like architecture with energy-channeling to resistive loads, providing a receiver that is configured as an absorber-as-film to capture electromagnetic energy after passing through the OUT. The metasurface may comprise spiral resonators to enable magnetic field dominant excitation, and thus the receiver antennas can be used for contrasting conductivity, Advantageously, the 2D metasurface reduces or avoids the need to linearly translate the metasurface to different positions along the longitudinal axis (i.e. the z-axis) of the object under test during imaging. In some embodiments, the height of the metasurface may be equal to or greater than the object under test, to thereby avoid the need to linearly translate the metasurface 300 along the longitudinal axis of the object under test during imaging. A width of the metasurface 300 (i.e. in the x-axis) may likewise be based on the size of the object under test.

It will be appreciated that the representation of the metasurface 300 shown in FIG. 3 is provided for the sake of explanation only and is non-limiting. In particular, the size of sensor array, including dimensions and number of receiver antennas, is non-limiting. As an alternative example, the metasurface may comprise a plurality of receiver antennas arranged in a line in the x-direction, and the receiver may be linearly translated along the longitudinal axis of the object under test in conjunction with the transmitter. Regardless of the particular metasurface size, in general the metasurface should comprise receiver antennas that have a periodic structure in order to perform calculations for periodic boundary conditions.

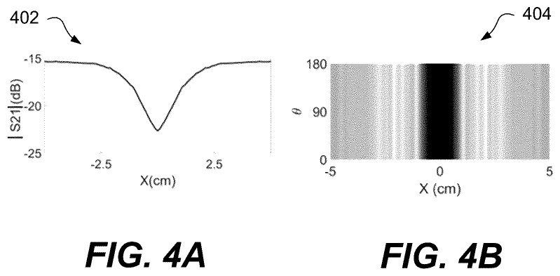

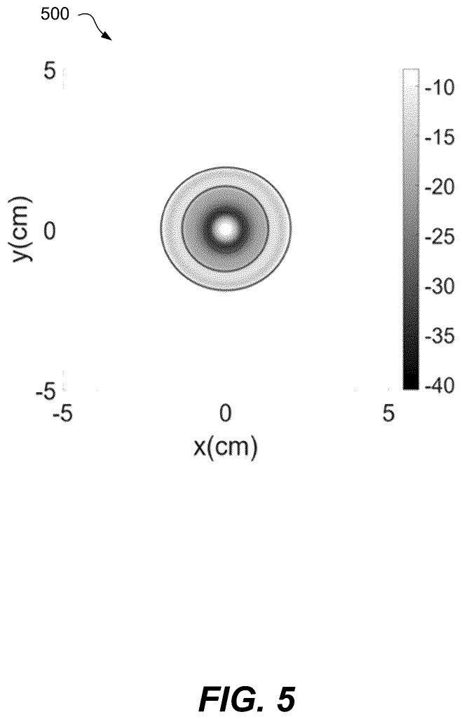

FIG. 4A shows a plot 402 of a back-projected profile of the object under test shown in FIG. 1. As described above, the transmitter antenna is configured to emit low frequency electromagnetic radiation towards the object under test, and the low frequency electromagnetic radiation received from the transmitter after passing through the object under test is recorded at a plurality of receiver antennas on the metasurface of the receiver. The plot 402 shown in FIG. 4A corresponds to a given imaging position with the transmitter antenna 102 and the receiver 104 arranged at opposite sides of the object under test. In the plot 402, the low frequency electromagnetic radiation is recorded at a line of the plurality of antennas that are in a plane (i.e. the x-y plane) comprising the transmitter antenna. The x-axis in the plot 402 represents an offset of a given receiver antenna in the x-axis direction with respect to the transmitter antenna. That is, a receiver antenna at x=0 is directly across from the transmitter antenna. The y-axis in the plot 402 is an RF parameter representing the amount of power transferred from the transmitter antenna to the receiver antenna. Signal processing algorithms are used to interpret the data collected from the antennas effectively, including filtering and performing back-projection.

As described above, once the data is collected for a given imaging position, the transmitter antenna and the receiver are rotated (or the object under test is rotated). Accordingly, in a given x-y plane along the longitudinal axis of the object under test, imaging is performed at different angles to collect data representing a full 360 degree coverage of the object under test. For each imaging position, a plot similar to the plot 402 shown in FIG. 4A can be produced, and then, from the data collected at each imaging position in a given x-y plane, a sinogram can be produced that represents the amount of power transferred from the transmitter antenna to the receiver antenna at different angles of rotation θ around the longitudinal axis of the object under test in that plane.

FIG. 4B shows a filtered sinogram 404 for the object under test shown in FIG. 1. The sinogram 404 shows the amount of power transferred from the transmitter antenna to the receiver antenna at different angles of rotation θ around the longitudinal axis of the object under test.

Similar profiles and sinograms of the object under test can be produced along the longitudinal axis of the object under test by linearly translating the transmitter antenna (and optionally the receiver antenna if required). Once the required data is collected from each imaging position to provide complete 360 degree coverage of the object under test along the longitudinal axis of the object under test, appropriate image processing algorithms are applied to reconstruct the image of the object under test.

Image reconstruction algorithms that can process the amplitude of transmission signals need to be robust enough to work without a heavy reliance on complex model-based reconstructions thereby ensuring computational efficiency. The application of the Radon transform for image reconstruction may be used, which is a mathematical technique that allows for the analysis of line integrals and the transformation of collected signal data into meaningful images, analogous to computerized tomography (CT).

Accordingly, back-projected graphs and the Radon Transform may be used to process the signals acquired from the receiver antennas, enabling accurate image formation of the object under examination. This methodology enhances the precision and resolution of the final reconstructed image by effectively integrating multiple perspectives into a coherent representation.

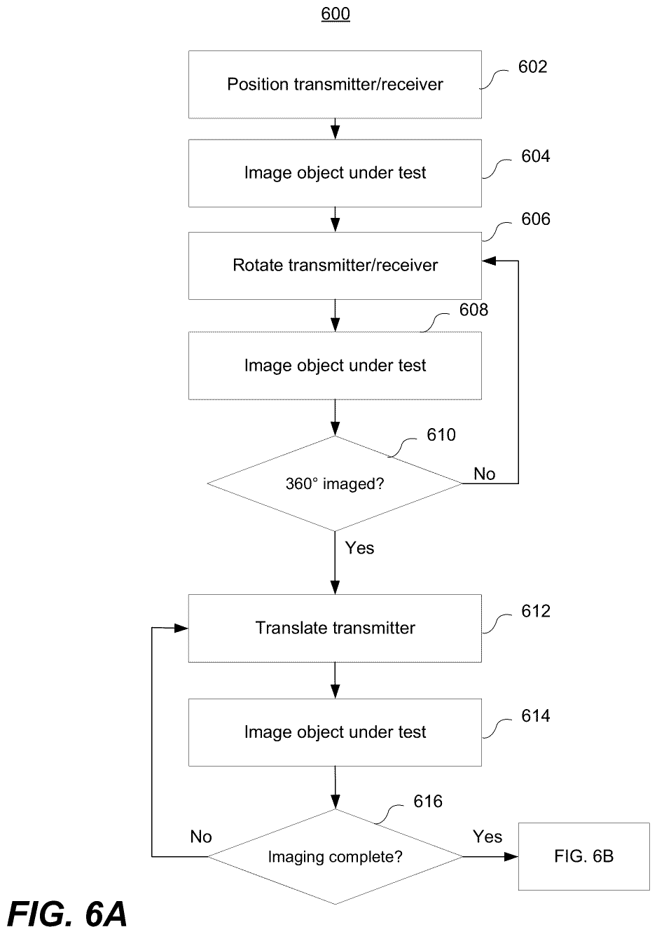

FIG. 5 shows a reconstructed image 500 of the object under test. In this example, the object under test is a four-layer 30-cm-long cylinder made up of two Plexiglas cylindrical shells. As seen in the reconstructed image 500, the outer shell has an outer radius of 1.6 cm and an inner one of 1.4 cm; the inner shell has an outer radius of 0.95 cm and an inner one of 0.85 cm; and the space between these two shells is filled with a mixture of sand and water with volumetric moisture, mᵥ, equals 20%. The space inside the inner shell is air.

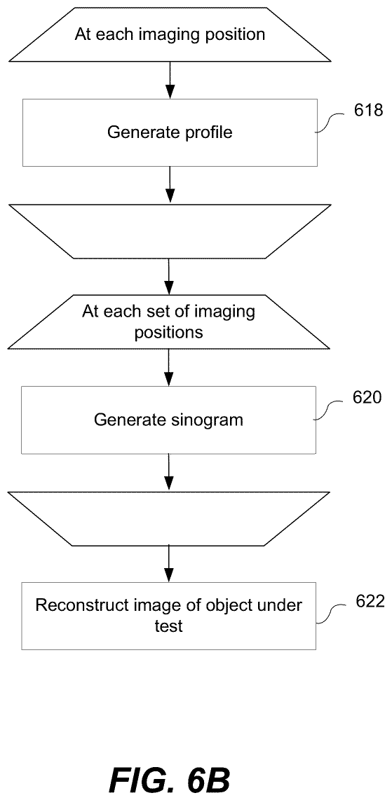

FIGS. 6A and 6B show a method 600 for performing computerized tomography of an object under test with low frequency electromagnetic radiation. A controller of the system may be used to control positioning and imaging functionality of the transmitter and receiver antennas, and to acquire data from the antennas. The method 600 may be stored as computer-executable instructions on a non-transitory computer-readable medium which, when executed by a processor, configures the controller to implement at least some aspects of the method 600. Further, the controller, or a computer device coupled with the controller, may be configured to process the data recorded from the antennas and generate a reconstructed image of the object under test.

The method 600 comprises positioning a transmitter antenna and a receiver on opposite sides of a longitudinal axis of the object under test in a first imaging position (602). In this position, the transmitter antenna is configured to emit low frequency electromagnetic radiation towards the object under test, and the receiver comprises a metasurface having a plurality of receiver antennas configured to receive the low frequency electromagnetic radiation emitted from the transmitter antenna after passing through the object under test.

The object under test is imaged at the first imaging position (604). Imaging is performed by emitting the low frequency electromagnetic radiation from the transmitter antenna toward the object under test, and recording, at the plurality of antennas, the low frequency electromagnetic radiation received from the transmitter after passing through the object under test. In particular, the low frequency electromagnetic radiation may be recorded at a line of the plurality of antennas that are in a plane comprising the transmitter antenna, the plane being perpendicular to the longitudinal axis of the object under test.

The transmitter antenna and the receiver are rotated about the longitudinal axis of the object under test to a second imaging position (606). The imaging of the object under test is performed at the second imaging position (608).

A determination is made if imaging has been performed 360 degrees about the longitudinal axis of the object under test (610). If not (NO at 610), the method returns to 606 and the transmitter antenna and the receiver are rotated and further imaging is performed. Once imaging has been performed 360 degrees about the longitudinal axis of the object under test (YES at 610), the transmitter antenna (and optionally the receiver if required) is translated in the longitudinal axis direction (612), and the imaging of the object under test is repeated (614) at further imaging positions for 360 degrees about the longitudinal axis of the object under test.

A determination is made if imaging is complete (616). That is, the determination is made whether the object under test has been imaged 360 degrees about the longitudinal axis of the object under test for the entire length/height of the object under test. If imaging is not complete (NO at 616), the transmitter antenna is translated in the longitudinal axis direction to another imaging position. If imaging is complete (YES at 616), the data recorded from the receiver antennas at each imaging position is used to reconstruct an image of the object under test, as described with reference to FIG. 6B.

Referring to FIG. 6B, at each imaging position, a profile of the object under test is generated (618), which may be similar to the profile shown in the plot in FIG. 4A. Then, for each set of imaging positions in a plane perpendicular to the longitudinal axis direction, a sinogram is generated (620), which may be similar to the sinogram shown in FIG. 4B. An image of the object under test is reconstructed based on the sinograms generated for each plane along the longitudinal axis of the object under test (622).

It will be appreciated that aspects of the method 600 may be performed simultaneously or in a different order than that shown in FIGS. 6A and 6B. For example, generating a profile of the object under test based on the low frequency electromagnetic radiation received at the plurality of antennas may be performed as soon as the imaging is done at a given imaging position (e.g. concurrently with or before rotating the transmitter and receiver). Likewise, generating a sinogram for each set of imaging positions in a plane may be performed as soon as the imaging is done in that plane (e.g. concurrently with or before linearly translating the transmitter along the longitudinal axis). Processing the data as it is collected may help to support real-time or near-real-time imaging of the object under test.

It would be appreciated by one of ordinary skill in the art that the system and components shown in the figures may include components not shown in the drawings. For simplicity and clarity of the illustration, elements in the figures are not necessarily to scale and are only schematic. It will be apparent to persons skilled in the art that a number of variations and modifications can be made without departing from the scope of the invention as described herein.

It is contemplated that any part of any aspect or embodiment discussed in this specification can be implemented or combined with any part of any other aspect or embodiment discussed in this specification.

It should be recognized that features and aspects of the various examples provided above can be combined into further examples that also fall within the scope of the present disclosure.

When used in this specification and claims, the terms “comprises” and “comprising” and variations thereof mean that the specified features, steps, or components are included. The terms are not to be interpreted to exclude the presence of other features, steps, or components.

The invention may also broadly consist in the parts, elements, steps, examples and/or features referred to or indicated in the specification individually or collectively in any and all combinations of two or more said parts, elements, steps, examples, and/or features. In particular, one or more features in any of the embodiments described herein may be combined with one or more features from any other embodiment(s) described herein.

1. A system for performing computerized tomography of an object under test using low frequency electromagnetic radiation, comprising:
a transmitter antenna configured to emit low frequency electromagnetic radiation towards the object under test; and
a receiver comprising a metasurface having a plurality of receiver antennas configured to receive the low frequency electromagnetic radiation emitted from the transmitter antenna after passing through the object under test.

2. The system of claim 1, wherein the metasurface is one dimensional or two dimensional.

3. The system of claim 1, wherein the metasurface is two dimensional and extends in a plane in parallel with a longitudinal axis of the object under test.

4. The system of claim 3, wherein a height of the metasurface is greater than or equal to a height of the object under test in a longitudinal axis direction.

5. The system of claim 4, wherein the transmitter antenna is linearly translatable with respect to the object under test in the longitudinal axis direction.

6. The system of claim 1, wherein the low frequency electromagnetic radiation emitted by the transmitter antenna is recorded at a line of the plurality of receiver antennas that are in a plane comprising the transmitter antenna, the plane being perpendicular to the longitudinal axis of the object under test.

7. The system of claim 1, wherein the transmitter antenna and the receiver are rotatable around the object under test.

8. The system of claim 1, wherein the object under test is rotatable with respect to the transmitter antenna and receiver.

9. The system of claim 1, wherein the transmitter is configured to emit the low frequency electromagnetic radiation at a frequency of less than 1 GHz.

10. The system of claim 1, wherein a size of each of the transmitter antenna and the plurality of receiver antennas is less than one-tenth a wavelength of the low frequency electromagnetic radiation.

11. The system of claim 1, wherein a size of each of the transmitter antenna and the plurality of receiver antennas is less than one-tenth a radius of the object under test.

12. A method for performing computerized tomography of an object under test using low frequency electromagnetic radiation, comprising:
positioning a transmitter antenna and a receiver on opposite sides of a longitudinal axis of the object under test in a first imaging position, wherein
the transmitter antenna is configured to emit low frequency electromagnetic radiation towards the object under test; and
the receiver comprises a metasurface having a plurality of receiver antennas configured to receive the low frequency electromagnetic radiation emitted from the transmitter antenna after passing through the object under test; and
imaging the object under test at the first imaging position by:
emitting the low frequency electromagnetic radiation from the transmitter antenna toward the object under test; and
recording, at the plurality of antennas, the low frequency electromagnetic radiation received from the transmitter antenna after passing through the object under test.

13. The method of claim 12, wherein the low frequency electromagnetic radiation is recorded at a line of the plurality of antennas that are in a plane comprising the transmitter antenna, the plane being perpendicular to the longitudinal axis of the object under test.

14. The method of claim 12, further comprising rotating the object under test about the longitudinal axis to a second imaging position, and performing the imaging of the object under test at the second imaging position.

15. The method of claim 12, further comprising rotating the transmitter antenna and the receiver about the longitudinal axis of the object under test to a second imaging position, and performing the imaging of the object under test at the second imaging position.

16. The method of claim 15, further comprising repeating the rotating and the imaging until the imaging is performed for 360 degrees about the longitudinal axis of the object under test.

17. The method of claim 12, further comprising translating the transmitter antenna in the longitudinal axis direction, and repeating the imaging of the object under test at further imaging positions for 360 degrees about the longitudinal axis of the object under test.

18. The method of claim 12, further comprising generating a profile of the object under test based on the low frequency electromagnetic radiation received at the plurality of antennas, at each imaging position.

19. The method of claim 18, further comprising generating a sinogram for each set of imaging positions in a plane perpendicular to the longitudinal axis direction.

20. The method of claim 19, further comprising reconstructing an image of the object under test based on the sinogram generated for each set of imaging positions.
